(19) United States
(12) Patent Application Publication
(10) Pub. No.: US 2022/0093237 A1
(43) Pub. Date: Mar. 24, 2022

(54) SYSTEMS, APPARATUS, AND METHODS FOR CLOUD-BASED ANIMAL REPRODUCTION INFORMATION SHARING

(71) Applicant: BREED EXCEL, INC., Silver Lake, KS (US)

(72) Inventor: John Schmidtlein, Ankeny, IA (US)

(21) Appl. No.: 17/448,622

(22) Filed: Sep. 23, 2021

Related U.S. Application Data
(60) Provisional application No. 62/706,995, filed on Sep. 23, 2020.

(51) Int. Cl. G16H 40/20; G06F 16/27; G16H 70/20; G06Q 10/10; G16H 10/60

(52) U.S. Cl. CPC G16H 40/20; G06F 16/27; G16H 10/60; G06Q 10/1093; G16H 70/20

(57) ABSTRACT

A cloud-based software can be utilized by both breeder and clinic or veterinarian. The breeder and clinic or veterinarian can share profiles for a specific canine or a group of canines. Information entered, such as progesterone results, will prompt automatic notification, email, and text messaging between users. Parameters will be set by clinic or breeder allowing the system to follow specific commands within the breeding process. At the end of the breeding process, a PDF summary will be automatically generated for export.

### CROSS REFERENCE TO RELATED APPLICATIONS

This application claims priority under 35 U.S.C. § 119 to provisional patent application U.S. Ser. No. 62/706,995, filed Sep. 23, 2020. The provisional patent application is herein incorporated by reference in its entirety, including without limitation, the specification, claims, and abstract, as well as any figures, tables, appendices, or drawings thereof.

### FIELD OF THE INVENTION

The invention relates generally to a system, apparatus, and/or corresponding method of use in at least the pet breeding, animal husbandry, and/or veterinary industries. More particularly, but not exclusively, the invention relates to cloud-based sharing of animal health and/or reproduction information.

### BACKGROUND OF THE INVENTION

Humans have maintained populations of useful animals around their places of habitat since pre-historic times. Over the years, animals have been domesticated for distinct purposes, including: meat and dairy consumption (e.g., cattle, pigs, goats, chicken, etc.), aids (e.g., guard, hunting, and guide dogs), sport (e.g., equestrianism), companionship, and the like.

To better effect these purposes, humans have intentionally mated some animals with certain characteristics to encourage those characteristics in the offspring. Through this process, hundreds of new breeds have been developed. Artificial selection during breeding has influenced behavior, shape, health, and size of animals.

Recent advancements in breeding technologies and an increase in the universal standard of living have allowed a large number of persons and entities to be able to breed animals. For example, breeding practices of kennels became much more rigorous and developed during the mid-19th century, which has led to the discovery of hundreds of new types of dog breeds. There are various reasons for people to breed animals. Some people may have personal reasons, such as wanting to own or give away animals, and are not interested in pedigree. Breeders may also breed animals for profit, for show, because of an interest in a particular breed, or to correct some issue and improve the soundness of a breed. Responsible breeders take into consideration the temperament, as well as the health and appearance of the mating pair before breeding.

For years breeders of purebred animals have been able to register the birth of litters to registries in order to record the parentage of the litter in stud books, such as those associated with kennel clubs. Such registries maintain records of animals' lineage. Maintaining correct data is important for breeding. Access to records allows a breeder to analyze the pedigrees and anticipate traits and behaviors that may be passed onto offspring. Requirements for the breeding of registered purebreds vary between breeds, countries, clubs, and registries. Breeders have to abide by the rules of the specific organization to participate in its breed maintenance and development programs. The rules may apply to the health of the animals and may owners to provide x-rays, certifications, and other proof of examinations related to the animals in order to comply with local regulatory authorities. North America in particular has noticeably less stringent requirements associated with the breeding of animals and/or mechanisms for enforcing these requirements. The quality of breeding practices is thus highly varied.

These breeding practices have not been without criticism. For example, modern dog breeding practices have been blamed for there being lower genetic diversity in animals than ever. This can lead to increased risk of widespread disease where breed-specific genetic traits are to blame for an animal's susceptibility to such disease. Dog breeding, if practiced irresponsibly, can also lead to pet overpopulation.

Increased awareness and access to reliable information can help pet owners, breeders, and veterinarians better fight these issues together. Thus, there exists a need in the art for a customizable cloud-based software application that can be intuitively and simultaneously utilized by animal owners, breeders, and veterinarians to better track all aspects of the breeding process.

### SUMMARY OF THE INVENTION

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part.

It is a primary object, feature, and/or advantage of the invention to improve on or overcome the deficiencies in the art.

It is a further object, feature, and/or advantage of the invention to solve problems necessarily rooted in computer technology.

It is a still further object, feature, and/or advantage of the invention to control movement of electromechanical equipment and/or components with software.

It is a still further object, feature, and/or advantage of the invention to process (e.g., qualitatively, quantitatively, and/or computationally), record, store, and/or link external technical data, such as image and/or video data.

It is a still further object, feature, and/or advantage of the invention to employ computer programs, libraries, user interfaces, integrated circuits, and/or algorithms specially adapted to better effect inventive concepts disclosed herein.

It is still yet a further object, feature, and/or advantage of the invention to automate data entry, decision making, the sharing of information relating to specific animals and/or groups of animals, breeding and/or veterinary tasks, and/or prompt notification, email, and text messaging between users. In some embodiments, it is preferred such automation is performed in real-time. In other embodiments, such automation will occur in response to specific, customizable input parameters.

The cloud-based software system disclosed herein can be used in a wide variety of applications. For example, veterinary and reproductive clinics can utilize the cloud-based software system to automate and grow the reproductive side of their business, while retail and hobby breeders can use the cloud-based software system to organize their breeding program.

It is preferred the cloud-based software system be cost effective and reliable. For example, access to the computerized system and/or certain information contained therein may be restricted, protected by encryption, and/or require the use of login credentials. Such security can help keep information private, if necessary, and may prove to be particularly important where breeders only choose to share negative information, such as health issues, with other parties they trust.

It is further preferred the cloud-based software system promote the safety and health of animals.

At least one embodiment disclosed herein contemplates the use of a distinct aesthetic appearance. Ornamental aspects included in such an embodiment can help capture a consumer's attention and/or identify a source of origin of a computerized product or process being sold. Said ornamental aspects will not impede functionality of the invention.

Methods can be practiced which facilitate use, manufacture, assembly, maintenance, and repair of components within the cloud-based software system which accomplish some or all of the previously stated objectives.

According to some aspects of the present disclosure, a computerized system for sharing breeding and/or animal related data among peers comprises a non-transitory computer readable medium having a processor, a memory, an operating system, and a compiler configured to communicate, analyze, and manage aspects related to animal breeding and/or health. A cloud-based storage system stores information related to said aspects. A software application can be executed by the non-transitory medium, is stored on the cloud-based storage system, and is configured to provide varying levels of access to said aspects depending on a type of user. An RFID-reader can be configured to identify RFID tags which include digital data identifying at least some of said aspects for the associated animal.

These and/or other objects, features, advantages, aspects, and/or embodiments will become apparent to those skilled in the art after reviewing the following brief and detailed descriptions of the drawings. Furthermore, the present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

### BRIEF DESCRIPTION OF THE DRAWINGS

Several embodiments in which the invention can be practiced are illustrated and described in detail, wherein like reference characters represent like components throughout the several views. The drawings are presented for exemplary purposes and may not be to scale unless otherwise indicated.

FIG. 1 illustrates, schematically, a hardware environment emphasizing computing components of an exemplary computing device, such as a desktop computer with separate devices responsible for accepting input and controlling audio and video output.

FIG. 2 exemplifies a cloud computing environment.

FIG. 3 depicts a dashboard of a software application according to some aspects of the invention.

FIG. 4 illustrates an exemplary profile associated with a canine.

FIG. 5 shows a decision tree illustrating automated aspects of methods described herein, the methods being responsive to customizable parameters which are set and relate to the breeding and/or care of animals.

The appendix shows screenshots of exemplary reports and/or graphical user interfaces which embody at least one aspect of the invention. In particular, pages A-B show an exemplary summary for a pet which has data related to a progesterone test and dates for artificial insemination, page C shows specific aspects related to access management, page D generically shows a dashboard, page E shows what aspects of a user profile can be edited, figure F relates to which parameters can be customized during artificial insemination, transcervical insemination, and surgical insemination; page G likewise shows parameters which can be customized for ultrasound tests, deworming, x-rays, and c-sections; page J likewise shows parameters which can be customized for progesterone tests; pages H-I shows specific aspects related to pet management; page K likewise shows specific aspects related to schedule management; pages M-N likewise relate to specific aspects related to user management and test setup; and page L exemplifies a specific test entry.

An artisan of ordinary skill need not view, within isolated figure(s), the near infinite number of distinct permutations of features described in the following detailed description to facilitate an understanding of the invention.

### DETAILED DESCRIPTION OF THE INVENTION

The present disclosure is not to be limited to that described herein. Mechanical, electrical, chemical, procedural, and/or other changes can be made without departing from the spirit and scope of the invention. No features shown or described are essential to permit basic operation of the invention unless otherwise indicated.

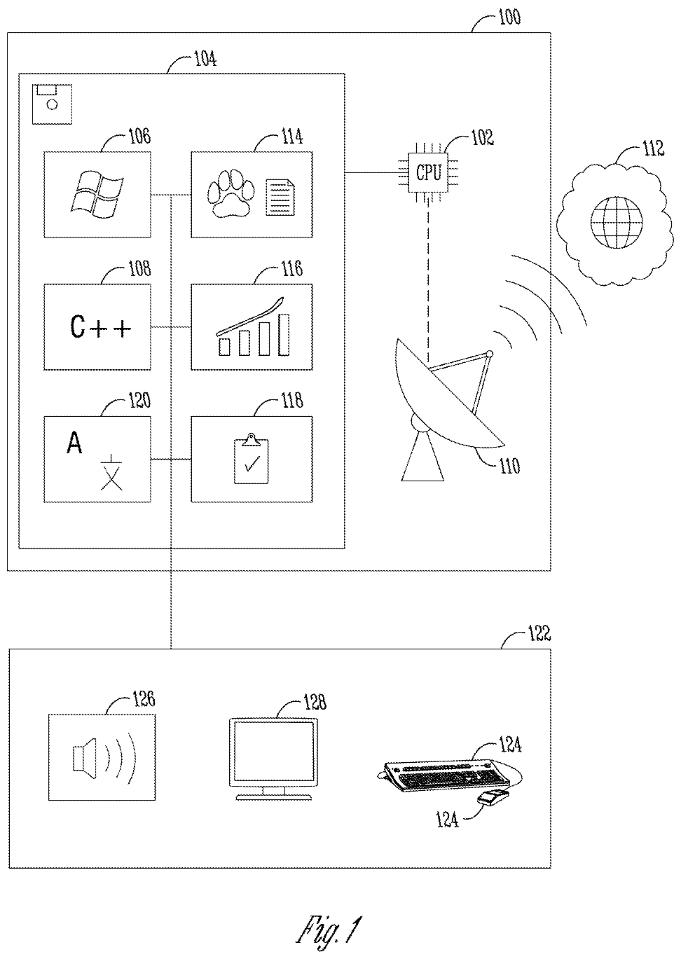

Referring now to FIGS. 1-4, exemplary hardware and software environments are shown through which the computerized, cloud-based system may be realized and/or used.

The user will typically interact with the computerized, cloud based system by way of a personal electronic device, such as a desktop computer, tablet, smartphone, gaming console, portable handheld device, laptop, smart device (TV, speaker, watch, etc.) and/or user display, however it is to be appreciated any suitable computing device can be used. Such personal electronic devices will preferably include, or will be remotely associated with, a non-transitory computer readable medium 100, such as that schematically depicted in FIG. 1.

The non-transitory computer readable medium 100 will preferably include an intelligent control (i.e., a controller) and components for establishing communications. Examples of such a controller may be processing units 102 alone or other subcomponents of computing devices. The controller can also include other components and can be implemented partially or entirely on a semiconductor (e.g., a field-programmable gate array (“FPGA”)) chip, such as a chip developed through a register transfer level (“RTL”) design process.

The processing unit 102, also called a processor, is an electronic circuit which performs operations on some external data source, usually memory 104 or some other data stream. Non-limiting examples of processors 102 include a microprocessor, a microcontroller, an arithmetic logic unit (“ALU”), and most notably, a central processing unit (“CPU”). A CPU, also called a central processor or main processor, is the electronic circuitry within a computer that carries out the instructions of a computer program by performing the basic arithmetic, logic, controlling, and input/output (“I/O”) operations specified by the instructions.

The memory 104 includes, in some embodiments, a program storage area and/or data storage area. The memory 104 can comprise read-only memory (“ROM”, an example of non-volatile memory, meaning it does not lose data when it is not connected to a power source) or random access memory (“RAM”, an example of volatile memory, meaning it will lose its data when not connected to a power source). Examples of volatile memory include static RAM (“SRAM”), dynamic RAM (“DRAM”), synchronous DRAM (“SDRAM”), etc.

Examples of non-volatile memory include electrically erasable programmable read only memory (“EEPROM”), flash memory, hard disks, SD cards, etc. In some embodiments, the processing unit 102 is connected to the memory 104 and executes software instructions that are capable of being stored in a RAM of the memory 104 (e.g., during execution), a ROM of the memory (e.g., on a generally permanent basis), or another non-transitory computer readable medium such as another memory or an external disc drive.

Generally, the non-transitory computer readable medium 100 operates under control of an operating system 106 stored in the memory 104. The non-transitory computer readable medium 100 implements a compiler 108 which allows a software application written in a programming language such as COBOL, C++, FORTRAN, or any other known programming language to be translated into code readable by the central processing unit 102. After completion, the central processing unit accesses and manipulates data stored in the memory 104 of the non-transitory computer readable medium using the relationships and logic dictated by the software application and generated using the compiler.

In one embodiment, the software application and the compiler are tangibly embodied in the non-transitory computer-readable medium 100. When the instructions are read and executed by the non-transitory computer readable medium 100, the non-transitory computer readable medium 100 performs the steps necessary to implement and/or use the invention. A software application, operating instructions, and/or firmware (semi-permanent software programmed into read-only memory) may also be tangibly embodied in the memory 104 and/or data communication devices 110, thereby making the software application a product or article of manufacture according to the invention.

A processor is an electronic circuit which performs operations on some external data source, usually memory or some other data stream. Non-limiting examples of processors include a microprocessor, a microcontroller, an arithmetic logic unit (“ALU”), and most notably, the central processing unit (“CPU”) 104. The CPU 104 carries out the instructions of a computer program by performing the basic arithmetic, logic, controlling, and input/output (“I/O”) operations specified by the instructions, such as those instructions specially adapted for managing aspects of animal breeding and health contained within modules 114-120.

A database is a structured set of data typically held in a computer. The database, as well as data and information contained therein, need not reside in a single physical or electronic location. For example, the database may reside, at least in part, on a local storage device, in an external hard drive, on a database server connected to a network, on a cloud-based storage system (multiple computing devices connected to the cloud-based network 112), in a distributed ledger (such as those commonly used with blockchain technology), or the like. The use of a distributed ledger is particularly advantageous where there is a high risk one person seeks to later edit and/or fabricate health and/or genetic data, as there exists no central authority which can be breached (“hacked”). In such embodiments, cryptographic keys and signatures may complement the use of the distributed ledger so as to increase security. Similarly, if some information is lost because of damage to a physical drive, there will be many copies of the information located on other physical drives.

The user interface 122, in particular, is how the user interacts with the non-transitory computer readable medium 100 and modules 114-120 contained therein. The user interface 122 can be a digital interface, a command-line interface, a graphical user interface (“GUI”), oral interface, virtual reality interface, or any other suitable way a user can interact with a machine (user-machine interface), or any combination thereof. For example, the user interface 122 can include a combination of digital and/or analog input/output devices or any other type of input/output device required to achieve a desired level of control and monitoring of the breeding/health data 208 and/or tasks. Input(s) received from input devices 124 can be sent to a microcontroller to control operational aspects of the intelligent control 100. Examples of input devices 124 include computer mice, keyboards, touchscreens, knobs, dials, switches, buttons, speakers, microphones, LIDAR, RADAR, etc. Examples of output devices include audio speakers 126, displays 128 for graphical user interfaces, light emitting diode (LED) indicators, etc. In at least one embodiment, graphical user interfaces 140 are capable of displaying and communicating animal data 208 in real time.

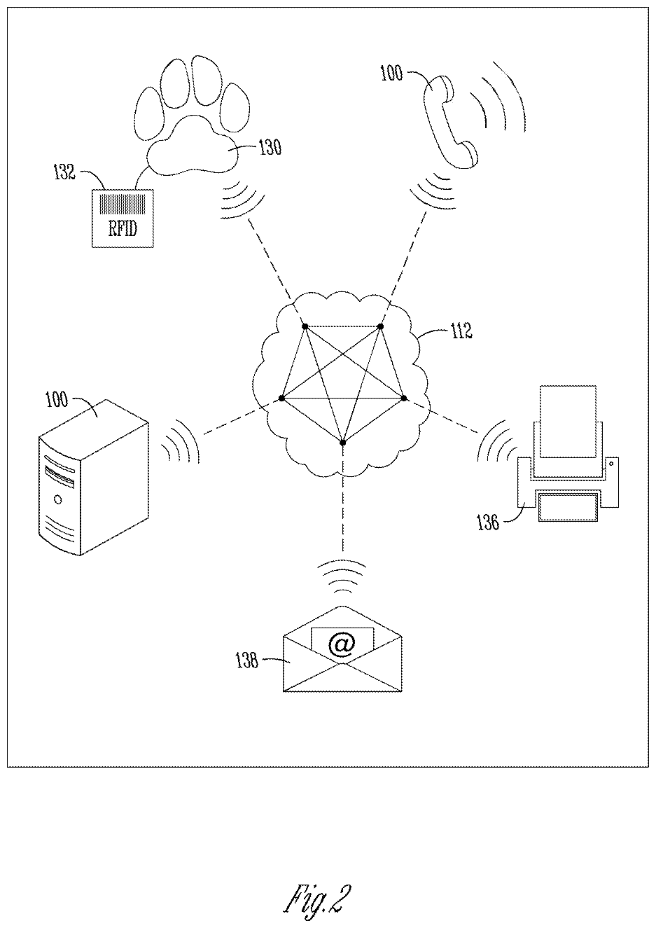

Referring now to FIG. 2, cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service.

A cloud computing environment is service oriented with a focus on statelessness, low coupling, modularity, and semantic interoperability. At the heart of cloud computing is an infrastructure comprising a network of interconnected nodes.

Exemplary characteristics of a cloud computing model follow. On-demand self-service: a cloud consumer can unilaterally provision computing capabilities, such as server time and network storage, as needed automatically without requiring human interaction with the service's provider. Broad network access: capabilities are available over a network and accessed through standard mechanisms that promote use by heterogeneous thin or thick client platforms (e.g., mobile phones, laptops, and PDAs). Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter). Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time. Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Exemplary service models of a cloud computing model follow. Software as a Service (SaaS): the capability provided to the consumer is to use the provider's applications running on a cloud infrastructure. The applications are accessible from various client devices through a thin client interface such as a web browser (e.g., web-based email). The consumer does not manage or control the underlying cloud infrastructure including network, servers, operating systems, storage, or even individual application capabilities, with the possible exception of limited user-specific application configuration settings. Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations. Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Exemplary deployment models of a cloud computing model follow. Private cloud: the cloud infrastructure is operated solely for an organization. It may be managed by the organization or a third party and may exist on-premises or off-premises. Community cloud: the cloud infrastructure is shared by several organizations and supports a specific community that has shared concerns (e.g., mission, security requirements, policy, and compliance considerations). It may be managed by the organizations or a third party and may exist on-premises or off-premises. Public cloud: the cloud infrastructure is made available to the general public or a large industry group and is owned by an organization selling cloud services. Hybrid cloud: the cloud infrastructure is a composition of two or more clouds (private, community, or public) that remain unique entities but are bound together by standardized or proprietary technology that enables data and application portability (e.g., cloud bursting for load balancing between clouds).

Here, the cloud computing network 112 and associated storage system can store and communicate genetic and/or health data of animals 130 with various types of non-transitory computer readable mediums 100 (e.g., the phone and desktop computer shown), computing devices attached to the animal 130, such as RFID tags 132 and/or scanners 134, wireless printers 136, email and/or notifications systems 138, and the like.

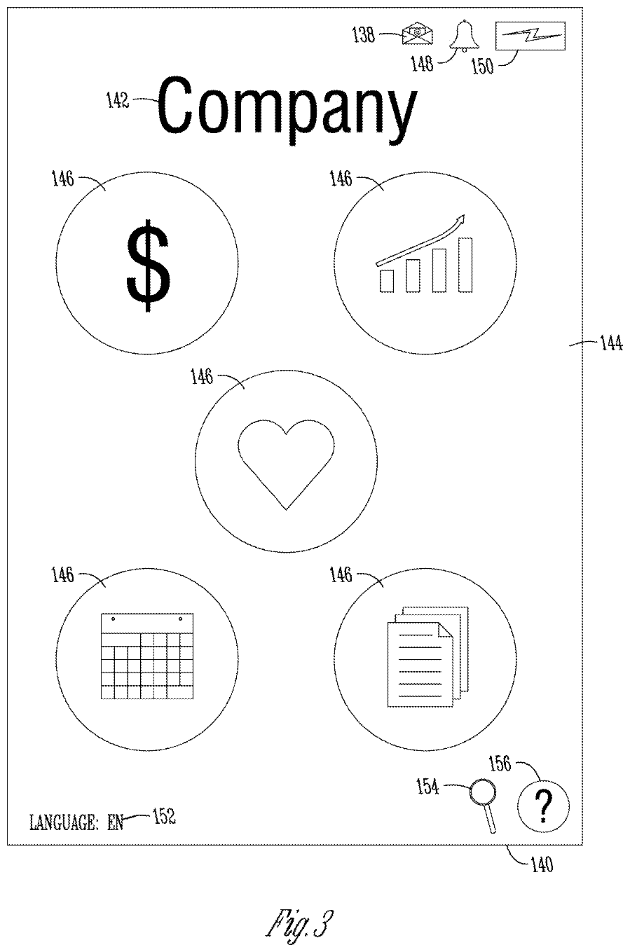

The graphical user interface 140 will typically include a source identifier 142 and/or logo to help the consumer identify which software application they are using. The graphical user interface will also include a dashboard 144 which acts as a main menu and provides the user the ability to engage each of the available functional aspects of the system and modules 114-120 by way of actions 146. Actions 146, can for example, allow the user to select and/or edit information in their profile or profiles associated with animals they own. Actions 146 can also allow the user to access records associated with animals (health/genetic information), generate and/or view customizable reports and/or pedigrees (progeny, siblings, ancestor analysis reports), maintain a calendar (including professional appointments, dated information related to an animal such as an animal's ovulation cycle, etc.), receive notifications 148, schedule updates, communicate by email 138, identify remaining battery life 150 of the non-transitory computer readable medium 100, select a language 152, search indices 154, and get help 156 (such as with tutorials, training options, FAQs, identification of corporate contact info, and the like). Clinics and commercial breeders may also be given access to actions which allow for the selection of waiting lists, business financials, trial mating's (genetic forecasting), etc., as is shown in FIG. 3.

Detailed information and parameters, such as breeder/clinic, can be stored in a breeder/clinic profile (not shown). In some embodiments, some aspects of the computerized system will be restricted until some fields within such a profile are completed. These fields can thus be thought to be “mandatory”. For example, to better determine what aspects of the system should be toggled and what regulations apply, a default language may have to be selected and a physical address entered. Other fields within the breeder/clinic profile may be designated as “optional”, such as the inclusion of mobile, work, and/or home phone numbers. Breeders and clinics may select whether they would like to retain the ability to automate the performance of tests and the receipt of notifications. This can be done, for example, by checking and unchecking boxes associated with the same. Once all the information is entered, the system will automatically generate a breeder/clinic unique identification number using which the breeder can search for the clinic.

Breeders may also be restricted such that they can view only certain information they have selectively been given access to. Such information may include, but is not limited to, pedigree numbers, names, gender, characteristics, health information, show results and inbreeding percentages of animals they own and/or animals they do not own, but are related to those animals they do own. In some embodiments, breeders are able to request mating information to help educate breeding decisions. In some embodiments, the requested information can then be used to create exemplary family trees, charts, and/or reports analyzing genetic probabilities of potential offspring can be created prior to mating. These types of documents may prove useful in more quickly gaining certifications from public and private authorities which regulate breeding.

Similarly, detailed information of a pet owner can be stored in a pet owner profile (not shown). Again, some of the information may be designated “mandatory” and some “optional”, and some information may be restricted from the user or owner's view. Once the details are initially completed and saved, the system will automatically generate a pet owner unique identification number using which the breeder/clinic can search the pet owner or pets linked to this pet owner.

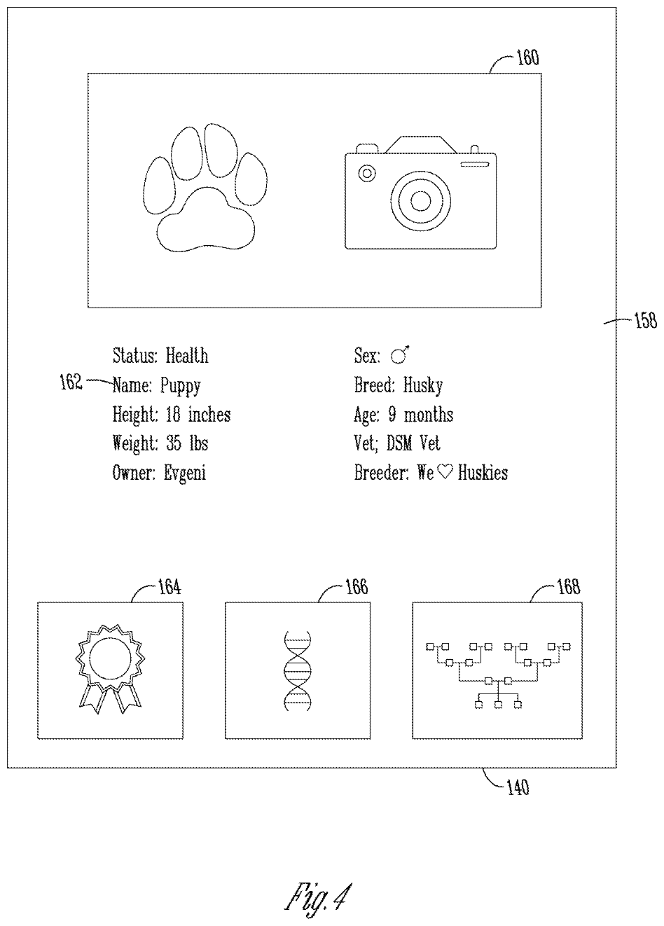

Animal profiles will include detailed health 162, genetic 166, and/or familial information 168. For example, for animal profiles a date of birth or age, coat color, height/length, weight, microchip number, DNA, and/or genetic testing information may be included, as shown in FIG. 4.

In some embodiments, some information, such as detailed health 162, genetic 166, and/or familial information 168 of an animal, will be viewable, however cannot be edited by, the pet owner. A breeder, veterinary clinic, vendor of the computerized system, Administrator, or other trusted, authorized user may be the only person(s) having access to such editing. In such embodiments, it is preferred the pet owner still retain the ability to request edits and/or certifications 164, pending approval of the authorized user. Still further, even commercial breeders may be restricted from some information and/or functionality reserved for veterinarians, such as the ability to schedule and perform tests like surgical insemination and c-sections. Where breeders are restricted from seeing and/or editing information and veterinarians are not, identical graphical user interfaces can be used, the exception being that inaccessible features are “grayed out.” Where this is not desirable, two distinct graphical user interfaces for breeder and veterinarian profiles would have to be created.

### Example 1: Generating Certifications and Reports

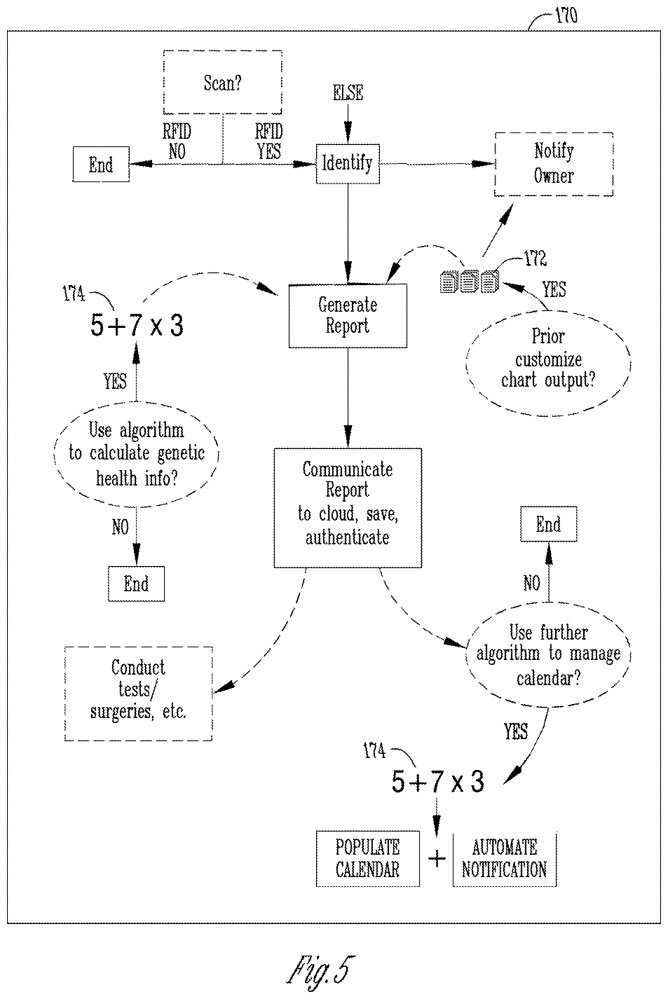

In a first example, one unique way in which the modules 114-120 may be utilized is to automatically generate certifications and reports periodically or upon request, as is depicted by the decision tree depicting the automated method 170 shown in FIG. 5. Further, as is descried in more detail with reference to EXAMPLE 2, automatic notifications and management of health/breeding related tasks can also be carried out using the same exemplary automated method 170.

First, it is preferred that the animal and/or person for which the animal is associated with be identified. To identify an animal, methods for maintaining identification and records relating to animals comprising the steps of obtaining information about an animal electronically can include implanting microchip technology having the read/write functionality within the animal, sensing a unique physical characteristic of the animal; and/or attaching an identifier (e.g. a tag) to the animal to manually identify the animal; and using RFID technology with a read/write functionality for storage of information. Such methods could be, as merely one example, those shown and described in U.S. Pat. No. 9,298,756, which is herein incorporated by reference in its entirety.

The owner, veterinarian, and/or breeder may then be notified upon the automatic identification and/or after generation of a report has occurred. This can be particularly advantageous where an animal is lost or where corporate entities are trying to track which animals must still be analyzed and/or tested. Such notification is optional and can be disabled where such a notification would be more burdensome than valuable.

Reports and charts 172 may include dam and sire records (dogs' basic info, names, age, breed, mating), litter record charts (birthing, siblings, weights, feeds, growth, treatments), microchip/contacts records (database and residency information), vaccination and health records (veterinary records), sales/adoption records (pricing, receipt, ownership identification, permit), socialization records (restrictions: does not socialize well with people/dogs/etc.).

Certifications will generally include the authority granting the certification. The certification can include signatures and/or watermarks if approved by said authority, which can be requested by the pet owner/breeder.

### Example 2: Docketing/Performing Health Related Tasks

Upon initial setup of profiles in the system, the system can automatically schedule and/or suggest dates for future appointments, health-related services, times to send reminders related to care for an animal, and the like. For example, the system can default to assigning responsibility for all tasks to a certified veterinarian and might not assign any such responsibilities to a commercial breeder. Such defaults may be altered if a particular relationship is established between veterinarian and breeder, or alternatively, the veterinarian may assign tasks once he or she determines in their professional expertise that responsibility for certain tasks may be delegated to commercial breeder and/or a pet owner. Some tasks, such as performing certain tests, might be unassignable, such that the risk of the veterinarian inadvertently assigns tasks reserved for the veterinarian is minimized.

The system's initial setup may also include a first, recommended list of options from which the veterinarian and/or other authorized user may customize. Such a list would include options which are regularly edited and/or require deference to the veterinarian's preferences. Further, other options which are substantially related to and/or are not usually separated but for extenuating circumstances, may automatically update upon editing some of these options. For example, if a veterinarian were to uncheck a ‘perform tests’ box, the box for automatically ‘receiving notifications’ might then be automatically unchecked. In some embodiments, the user will be able to see this automatic action, while in others, the user might have to open a second, advanced setup list which includes all manually editable options. This second list would include other options which are not usually altered, but could, for example, if the user had highly specific preferences or requirements.

Initial parameters and ranges of values specific to certain tests are all customizable and are not meant to be needlessly constrained. For example, these parameters and ranges are typically influenced only by a breeder's personal breeding practices and/or preferences, but can also be influenced by breeding/mating/animal health laws and regulations if there are any. By way of example only, customer relationship management (“CRM”) may be managed and initial parameters set as follows.

Breeding Profiles

Female and male canine profiles will include date of birth, color, DNA, height, length, weight, microchip number, genetic testing information, etc.

A female canine profile will note the first day of a female having a bloody discharge (considered to be day 1). Day 9 will be designated as the day upon which a first progesterone test is conducted. If the results are less than 2.5 ng/ml, the computerized system will automatically retest every 2 days, starting with day 11, until a value of 2.5 ng/ml or greater is realized. After realizing a value of greater than 2.5 ng/ml, daily tests will continue until the female hits ovulation at 5 ng/ml-8 ng/ml. Following confirmation of ovulation at 5 ng/ml-8 ng/ml, procedures are timed as follows: artificial insemination (“AI”): 48 hours following ovulation and again 96 hours following ovulation; transcervical insemination (“TI”): 48 hours following ovulation and again 96 hours following ovulation; surgical insemination (“SI”): 72 hours following ovulation. Each breeder has their own preference in which insemination methods they use, so this will need to be a choice selection. For example, the day ovulation starts may be defined as “day 1.” Day 28 or after, it may be suggested and/or scheduled to conduct an ultrasound after a pregnancy is confirmed. Veterinarians may be notified of potential due date for c-sections. On days 54-56, deworming may be scheduled. On day 55 or thereafter, x-rays can be taken to verify puppy count in the litter. On day 61, testing can begin for reverse progesterone to time c-sections. The due date generally falls on day 63.

A male canine profile will generally note volume and sperm analysis, including the count, morphology, and mobility associated therewith.

Automated schedules for canine puppies can be as follows: 1 week—send in DNA; 2 weeks—first deworming, Fenbendazole×3 days; 4 weeks—second deworming, Fenbendazole×3 days and Toltrazuril×1 day; 5 weeks—register litter; 6 weeks—third deworming, Fenbendazole×3 days and Toltrazuril×1 day; 6 weeks—veterinarian check-ups and vaccination: emails/texts to pet owners for reminding of the same, and these emails and texts can include next date for vaccination which will be 3 weeks following; 7 weeks—bring the puppy home and send reminder to buy: (a) playpen; (b) crate; (c) bowls (food bowl must be shallow); (d) harness/leash (breeder can input size); (e) name tag; (f) potty pads; (g) beds and blankets; (h) food—brands will differ from breeder; and (i) guides and/or manuals for properly caring for the canine; and 8 weeks-4th deworming, Panacur×3 days.

Switching food should be gradual and mixed with previous foods and/or canned pumpkins at first. This may help the puppies' digestion. Puppies may also be given single ingredient treats such as bully sticks, dried chicken feet, or sweet potatoes.

Dog Maintenance

Vaccinations should be prompted yearly. Fecals should be prompted yearly for deworming. Reminders should be sent monthly for flea/tick/heartworms. Grooming can be based on personal preference. Teeth cleaning will automatically depend on the dog's breed, else can be a yearly reminder that can be skipped.

Litter Reminders

Breeders may be reminded to check with pet home buyers after puppy is 7 months to be sure they have been spayed/neutered. Breeders may also be sent yearly birthday e-cards.

Pet Owners

A puppy health checkup can be set up for the first 3 days after puppy's arrival; Fecal can be checked at this appointment, prompting the owner to take a stool sample to appointment. Owners may be prompted to obtain pet health insurance as soon as they receive a puppy. Vaccinations can be scheduled 3 weeks following initial vaccination with booster, then again 3 weeks following. Pet owners will be prompted to spay/neuter at 7 months, however, could vary depending on contract with breeder. Vaccinations are to be prompted yearly following last vaccination, as are fecals for deworming. Reminders will be sent to check for flea/tick/heartworms on a monthly basis. Grooming can be set based on a personal preference. Teeth cleaning will automatically depend on the dog's breed, else can be a yearly reminder that can be skipped.

Supplies

Owners of supplies may be prompted to restock when low and/or when anticipated to be low. Said supplies may include: food; dewormers: Fenbendazole, Toltrazuril; components used during AI, including: catheters, extenders, lubricants, gloves, and tubes; semen shipments; and boxes.

Miscellaneous

Litter DNA predictions can be generated upon entering DNA results of female and males.

Appointments may be automatically set through the software application where pet owner, breeder, and/or veterinarian are already paired.

In yet another example, automated tests for canines may be conducted as follows:

If “Perform Test” is checked, then all the fields related to that test will be enabled for editing. If “Perform Test” is unchecked, all the fields associated with the appropriate test will be uneditable.

Progesterone Test

The same “ovulation method” selected using the dropdown is used throughout the application for that breeder/clinic. The breeder/clinic will have to select the ‘Ovulation method type’ from the dropdown which is ‘ng/ml’, ‘ng/dl’ and ‘nmol/l’. The default would be ‘ng/ml’.

Step 1 goal of progesterone test should be entered manually. However, the default value would be 1 ng/ml. Here, the step 1 test frequency and step 1 notification days before should be entered manually.

Step 1 test frequency is the number of days for performing progesterone test after the bloody discharge date. The default value is 5 days after bloody discharge.

Step 1 notification days before is the number of days prior to the step 1 test date. The default value is 2 days before the test date. The notifications will start going out 2 days before the test.

Step 2 goal of progesterone test should be entered manually. However, the default value would be 2.5 ng/ml. Here, the step 2 test frequency and step 2 notification days before should be entered manually.

Step 2 test frequency is the number of days for performing progesterone test after achieving step 1 goal. The default value is 2 days after achieving step 1 goal.

Step 2 notification days before is the number of days prior to the step 2 test date. The default value is 1 day before the test date. The notifications will start going out 1 day before the test.

Step 3 goal of progesterone test should be entered manually. However, the default value would be 5 ng/ml. Here, the step 3 test frequency and step 3 notification days before should be entered manually.

Step 3 test frequency is the number of days for performing progesterone test after achieving step 2 goal. The default value is 1 day after achieving step 2 goal

Step 3 notification days before is the number of days prior to the step 3 test date. The default value is 0 day before the test date. The notifications will start going out 0 day before the test.

Similarly, if the breeder/clinic has step 4 and step 5, it should be entered accordingly. If a breeder/clinic only follows 3 steps, then they need to leave the “Step 4 Goal” and “Step 5 Goal” blank. This way the system will understand that there are only 3 steps in the process.

Insemination Based on the insemination checked, the breeder/clinic will have to enter the hours of breeding after the ovulation date. AI has 3 breedings, TI has 2 breedings and SI has 1 breeding. The default values would be: AI Breeding 1: 0 hours after ovulation date; AI Breeding 2: 48 hours after ovulation date; AI Breeding 3: 96 hours after ovulation date; TI Breeding 1: 48 hours after ovulation date; TI Breeding 2: 96 hours after ovulation date; and SI Breeding 1; 72 hours after ovulation date.

Ultrasound. The breeder/clinic will have to enter the days of ultrasound after the ovulation date and notification days before ultrasound. Days of ultrasound after the ovulation date is the number of days for performing ultrasound after ovulation date. The default value is 28 days after ovulation date. Notification days before ultrasound is the number of days prior to the ultrasound date. The default value is 7 days before the ultrasound. The notifications will start going out7 days before the test.

Deworming

The breeder/clinic will have to enter the days of deworming after the ovulation date and notification days before deworming. Days of deworming after the ovulation date is the number of days for performing deworming after ovulation date. The default value is 55 days after ovulation date. Notification days before deworming is the number of days prior to the deworming. The default value is 2 days before deworming. The notifications will start going out2 days before deworming.

X-Ray

The breeder/clinic will have to enter the days of x-ray after the ovulation date and notification days before X-ray. Days of X-ray after the ovulation date is the number of days for performing X-ray after ovulation date. The default value is 55 days after ovulation date. Notification days before X-ray is the number of days prior to the X-ray. The default value is 7 days before X-ray. The notifications will start going out 7 days before X-ray.

C-Section

The Clinic will have to enter the days of C-section after the ovulation date and notification days before C-section. Days of C-section after the ovulation date is the number of days for performing C-section after ovulation date. The default value is 63 days after ovulation date. Notification days before C-section is the number of days prior to the C-section. The default value is 5 days before C-section. The notifications will start going out 5 days before C-section.

Semen Analysis

A semen analysis, also called seminogram, or spermiogram evaluates certain characteristics of a male's semen and the sperm contained therein, such as to evaluate male fertility. Depending on the measurement method, just a few characteristics may be evaluated (such as with a home kit) or many characteristics may be evaluated (generally by a diagnostic laboratory). Collection techniques and precise measurement method may influence results. A routine semen analysis should include the following data and/or parameters: physical characteristics of semen (color, odor, pH, viscosity and liquefaction), volume, concentration, morphology and sperm motility and progression.

Exemplary breeding test processes follow.

A female dog typically has two bloody discharges every year (once in 6 months) during which they are ready to breed. The bloody discharge date is considered to be the start date of the breeding process and this date should be the first thing to enter into the system. The system will then start working as per the values entered by the breeder during the registration. However, the system will have default values which the breeder can edit (while or after registration). All entries made for a particular pet, until the date of changes, will use old values and, for any future tests, will use new values.

For this process, we use the following values from breeder entity and the process entity. The values from the breeder entity which will be used are: ovulation method type (ng/ml, ng/dl or nmol/l)—for example ng/ml; PT maximum test (even though we are giving option till 5, this fields needs to be populated with the maximum number that breeder is using); PT test step 1 goal (e.g., value 1); PT test step 1 frequency (after 5 days); PT test step 1 notification days (2 days before); and similar entries for step 2-5.

The values from the process entity are based on the pet profile: microchip number, breeder number, test type (1-5), progesterone test method, progesterone test goal, step 1 test frequency, step 1 notification days, progesterone test date, notification date, and ovulation value.

The progesterone test should be performed on step 1 frequency date from the date of bloody discharge. The reminder for the progesterone test should be sent out from bloody discharge date+step 1 test frequency minus step 1 notification days and when the associated ovulation value is not populated. For example:

Bloody discharge date: Dec. 15, 2019

Progesterone Test Results

Microchip number: #12345

Breeder number: ID 45678A

Test type: step 1

Progesterone test date: Dec. 20, 2019

Progesterone test method: ng/ml

Progesterone test goal: 1

Ovulation value:

Notification date: Dec. 18, 2019 (Bloody discharge date (Dec. 15, 2019)+step 1 test frequency days (5) —step 1 notification frequency days (2)).

If the breeder has opted to receive notifications for progesterone test, the notifications should be sent out via email and also when the breeder logs into the accounts.

For example, if the bloody discharge had started on Dec 15^{th}, a reminder saying “Progesterone test scheduled for December 20^{th}” should start going out from December 18^{th}. If the step 1 Ovulation value is not populated and the test date has been passed, a red alert notification should show up when the breeder logs in.

Step 1—Ovulation Method ID:

PT step 1 goal—1 ng/ml

PT step 1 test frequency—5 days

PT step 1 notification days—2 days

When the user enters the bloody discharge is entered, the step 1 data for the process is automatically populated (except for ovulation value).

On 20th December, if the ovulation value entered is 0.08 ng/ml (which is less than step 1 goal), the system should recognize and automatically schedule the step 1 retest as per step 1 test frequency days by creating another record for step 1 for retesting. Here, the date for the retest should be auto populated and the ovulation value should be blank. Also, the notifications should be sent out based on the step 1 test frequency minus notification days.

Likewise, until the goal of step 1 is achieved, the retest dates should be auto populated with empty fields for ovulation value by creating records for each and every tests.

Once the goal of step 1 is achieved, another record is created for step 2 again with ovulation value not populated. The same thing is continued for step 2 and step 3, until we reach maximum steps (PT maximum steps) for that breeder.

If all the ovulation values are populated for a pet, then the progesterone test is complete.

In this case, a step 3 goal is achieved on 01-10-2020. Hence, the progesterone test result is 6.4 ng/ml achieved on January 10^{th} which is considered as final date of ovulation. Both these should be stored in the process at the pet level.

Insemination

There are 3 types of insemination which is different from each other. If the breeder has opted to receive notifications for insemination, the notifications should be sent out.

All fields should be auto populated. Reminders should be provided on a daily basis for all tests. AI (three breedings) (only done by breeder); TI (two breedings) (can be done by breeder or clinic); and SI (one breeding) (only done by clinic).

If the breeder has opted for AI, then the following process needs to be followed.

Each breeder can perform a maximum of 3 shots for a pet. Each breeder may perform shots at a different time. For example, a breeder can perform first shot on the day of ovulation, followed by the second shot at 48 hours after ovulation and the third at 96 hours after ovulation. Another breeder might do this differently. Since these values are not fixed, this information can be stored at the breeder level and for each pet, data retrieved from the breeder level and appropriate actions performed.

The values from the breeder entity which will be used are: first shot hours (for example, 0 hours after final date of ovulation), second shot hours (for example, 48 hours after final date of ovulation), and third shot hours (for example, 96 hours after final date of ovulation).

The values from the process entity is: microchip number, breeder number, final date of ovulation, first shot date and time, second shot date and time, and third shot date and time. Notifications for insemination should go out on a daily basis until the third shot is performed.

If AI is selected as the insemination type, the first breeding date may precede the second breeding date by two days, and the second breeding date may precede third breeding date by two days, etc. The breeder can enter the shot date and time from the calendar. Similarly, if TI is selected as the insemination type, then the dates should be populated accordingly.

Ultrasound Date

The values from the breeder entity which will be used are: ultrasound to be performed days after final ovulation date (e.g., 28); days to notify before ultrasound; the values from the process entity are (is for a pet); microchip number, ultrasound date; pregnancy result (positive/negative, the default being “select”).

From the breeder profile the system will know, if the ultrasound can be performed by the breeder or not. If the breeder can perform ultrasound, then the following process.

Based on the breeder profile “ultrasound to be performed days after final ovulation date “the system will know when to perform the ultrasound.” The ultrasound can be done on the 28^{th} day after the date of ovulation. If the breeder has opted to receive notifications for the ultrasound, the notifications should be sent out again based on the breeder profile. The ultrasound date field should be auto populated.

The only way the system understands, if the ultrasound is performed or not, is based on the “pregnancy test result.” This needs to be populated as soon as the results of ultrasound are available. This should be a check box.

If the result is negative, the values from the breeder entity which will be used are potential due date from the final day of ovulation in days (e.g., 63).

If pregnant, send notification to the clinic/vet about the potential c-section. This will be based on the “potential due date from the final day of ovulation in days”. Add the number of days in this field, to the Final Ovulation date and send an email.

Deworming Date

The values from the breeder entity which will be used are: number of days after ovulation for deworming (e.g., 54) and number of notifications days before deworming.

Based on the breeder profile, “No. of days after ovulation for deworming”, the system will know when to perform deworming. Again, this can be done 54 days from the date of “final ovulation date.” Based on the number of days before to notify about deworming, the notification is sent out.

In the process entity, the deworming date is automatically populated. The only way the system understands, if the deworming is performed on not, is based on the “deworming performed” check box. This needs to be populated as soon as the deworming is completed.

X-Ray Date

The values from the breeder entity which will be used are: number of days after ovulation for X-ray (e.g., 55) and number of notification days before the X-ray.

Based on the breeder profile, “No. of days after ovulation for X-ray” the system will know when to perform X-ray. In our example, it is done after 55 days from the date of “final ovulation date.” Based on the number of days before to notify about X-ray, the notification is sent out.

In the process entity, the X-ray date is automatically populated. The only way the system understands, if the X-ray is performed on not, is based on the “X-ray performed” check box. This needs to be populated as soon as the X-ray is completed. Moreover, there will also be a field called puppy count, which needs to be populated after the X-ray is completed.

Similar information related to the following processes: reverse progesterone tests and due date, are consistent with the principles put forth herein and can be automated.

Since the entire process is long and complicated, in the female pet profile, there may need to be a field which clearly states what stage of breeding is the pet in. This will help the program to run much faster to exactly go to the right process. Every time the process changes, this field needs to be updated automatically by the system. Once the pregnancy is complete, the breeder can mark it as complete.

From the foregoing, it can be understood that the invention accomplishes at least all of the stated objectives.

Unless defined otherwise, all technical and scientific terms used above have the same meaning as commonly understood by one of ordinary skill in the art to which embodiments of the invention pertain.

The terms “a,” “an,” and “the” include both singular and plural referents.

The term “or” is synonymous with “and/or” and means any one member or combination of members of a particular list.

The terms “invention” or “invention” are not intended to refer to any single embodiment of the particular invention but encompass all possible embodiments as described in the specification and the claims.

The term “about” as used herein refer to slight variations in numerical quantities with respect to any quantifiable variable. Inadvertent error can occur, for example, through use of typical measuring techniques or equipment or from differences in the manufacture, source, or purity of components.

The term “substantially” refers to a great or significant extent. “Substantially” can thus refer to a plurality, majority, and/or a supermajority of said quantifiable variable, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

In communications and computing, a computer readable medium is a medium capable of storing data in a format readable by a mechanical device. The term “non-transitory” is used herein to refer to computer readable media (“CRM”) that store data for short periods or in the presence of power such as a memory device.

One or more embodiments described herein can be implemented using programmatic modules, engines, or components. A programmatic module, engine, or component can include a program, a sub-routine, a portion of a program, or a software component or a hardware component capable of performing one or more stated tasks or functions. A module or component can exist on a hardware component independently of other modules or components. Alternatively, a module or component can be a shared element or process of other modules, programs, or machines.

Radio-frequency identification (“RFID”) relates to the use of electromagnetic fields to automatically identify and track tags attached to objects.

Artificial insemination (“AI”) as used herein is the deliberate introduction of sperm into a female's cervix or uterine cavity for the purpose of achieving a pregnancy through in vivo fertilization by means other than sexual intercourse.

Transcervical insemination (“TI”) as used herein is a non-surgical, non-invasive insemination technique whereby a catheter is passed through the cervix into the uterus using a specialized rigid endoscope. The semen is then flushed through the cervix and deposited directly into the uterus.

Surgical insemination (“SI”) as used herein refers to intra-uterine implantation of semen. SI is typically only used where semen is frozen or the quality of the semen is poor. Because the sperm is placed closer to the site of fertilization, this technique is associated with the greatest chance of conception however is a more invasive procedure than TI.

The “scope” of the invention is defined by the appended claims, along with the full scope of equivalents to which such claims are entitled. The scope of the invention is further qualified as including any possible modification to any of the aspects and/or embodiments disclosed herein which would result in other embodiments, combinations, subcombinations, or the like that would be obvious to those skilled in the art.

1. A computerized system for sharing breeding and/or animal related data among peers comprising:
a non-transitory computer readable medium comprising a processor, a memory, an operating system, and a compiler configured to communicate, analyze, and manage aspects related to animal breeding and/or health;
a cloud-based storage system for storing information related to said aspects;
a software application executable by the non-transitory medium and stored on the cloud-based storage system; and
a sensor for identifying an animal based upon a physical characteristic or an identifier associated with the animal;
wherein the software application is configured to:
provide varying levels of access to said aspects depending on a type of user;
automatically calculate and/or schedule tests, appointments, services, and/or health related tasks based on customizable input parameters specific to the breeding and/or care of the animal; and
retrieve digital data related to at least some of said aspects for the associated animal subsequent to the sensor identifying the animal.

2. The computerized system of claim 1, wherein the cloud-based database utilizes a distributed ledger.

3. The computerized system of claim 1, wherein tasks related to caring for the associated animal are automatically assigned based on the type of user, said type of user optionally selected from the group consisting of: veterinarian, breeder, and owner.

4. The computerized system of claim 1, further comprising a display capable of displaying a user interface through which a user interacts with the computerized system, said graphical user interface allowing the user options (i) to edit a profile associated with the user and/or animals associated with the user, (ii) view health and/or genetic records, (iii) generate customizable reports and/or pedigrees; and (iv) access a calendar.

5. The computerized system of claim 4, wherein the user interface includes a virtual assistant with voice recognition capabilities.

6. The computerized system of claim 4, wherein the user interface works in connection with a means for audio output to notify the user of alerts and/or upcoming events.

7. The computerized system of claim 4, wherein the user interface includes means for assisting the user selected from the group consisting of: instructions; tutorials; training services; frequently asked questions; corporate contact information; and searchable indices to aid in navigating the user interface.

8. The computerized system of claim 1, wherein the digital data is communicated and said communicated digital data is encrypted.

9. The computerized system of claim 1, further comprising a wireless printer for printing reports, charts, and/or certificates generated by the non-transitory computer readable medium and software application.

10. The computerized system of claim 1, wherein aspects of the computerized system are restricted until at least two types of users complete all fields designated mandatory of user profiles relating to an associated animal and/or group of associated animals and/or all required input parameters are assigned an initial value.

11. The computerized system of claim 1, wherein the software application is further configured to analyze potential mating's.

12. The computerized system of claim 1, wherein the identifier is an RFID tag or a microchip.

13. A method for authenticating identification of animals comprising:
storing aspects related to animal breeding and/or health on a cloud-based storage system;
customizing said aspects to include at least a health condition of an animal or a process for caring for the animal;
communicating, via a distributed ledger, said aspects to at least two different types of users; and
restricting further editing of some of the aspects based upon the type of user.

14. The method of claim 13, further comprising scanning a barcode to effect identification of a lost animal.

15. The method of claim 13, further comprising electronically generating reports, charts, and/or certificates related to the associated animal.

16. The method of claim 15, further comprising requesting a professional person and/or organization to certify, authenticate, and/or sign said reports, charts, and/or certificates.

17. A method of managing health and/or genetic information of animals comprising:
storing aspects related to animal breeding and/or health on a cloud-based storage system;
automatically scheduling appointments, reminders, and/or tasks to be performed based on initial data entered into the cloud-based storage system, said initial data identifying at least a type of user; and
notifying person(s) of said appointments, reminders, and/or tasks.

18. The method of claim 17, wherein the appointments, reminders, and/or tasks relate to a procedure selected from the group consisting of insemination, testing for progesterone, performing an ultra-sound, deworming, and taking x-rays.

19. The method of claim 17, wherein some of said aspects relate to commercial endeavors, said some of said aspects can only be viewed by appropriate types of users, and, optionally, said some of said aspects relate to waiting/customer lists, track and analyze business or clinic financials, or integrate said appointments, reminders, and/or tasks to a professional's existing calendar.

20. The method of claim 17, further comprising advertising services which better promote the health of animals, such as adoption and/or fostering of the same, to existing users of the cloud-based storage system.
